(19) United States
(12) Patent Application Publication
(10) Pub. No.: US 2025/0223399 A1
(43) Pub. Date: Jul. 10, 2025

(54) PREPARATION OF A COMPOSITION COMPRISING AN ORGANIC PEROXIDE BY SOLVENT TRANSFER

(71) Applicant: Arkema France, Colombes (FR)

(72) Inventor: Serge HUB, Pierre-Benite Cedex (FR)

(21) Appl. No.: 18/856,248

(22) PCT Filed: Dec. 18, 2019
(86) PCT No.: PCT/FR2019/053172

§ 371 Date: Oct. 11, 2024

(30) Foreign Application Priority Data
Dec. 19, 2018 (FR) 1873432

(51) Int. Cl. C08G 63/78

(52) U.S. Cl. CPC C08G 63/78

(57) ABSTRACT

The present invention relates to a method for preparing a mixture comprising an organic peroxide and an additional compound by transferring said organic peroxide of a solvent into a composition comprising the additional compound.

### FIELD OF THE INVENTION

The present invention relates to a process for preparing a mixture comprising an organic peroxide and an additional compound by transferring said organic peroxide from a solvent into a composition comprising the additional compound.

### TECHNICAL BACKGROUND

Organic peroxides are very widely used in the chemical industry, in particular in the manufacture of plastics and rubber. They intervene as initiators in the radical polymerization of monomers into thermoplastic polymers, as hardeners for thermosetting polyester resins and as crosslinking agents for elastomers and polyethylene. In addition, organic peroxides serve as a source of free radicals in many organic syntheses.

Organic peroxides are very unstable species. They are often explosive and flammable compounds that break down violently, making them difficult to handle. Organic peroxides can also be corrosive and toxic. In order to limit the dangers and to facilitate their handling, organic peroxides are often handled in solvents.

For example, the document “Esters of Peroxycarbonic Acids” (Journal of American Chemical Society, volume 72, page 1254) describes the synthesis of monoperoxycarbonates and also dialkyl peroxydicarbonates using a volatile organic solvent. The organic solvent is used to solve a solid-handling problem, and it is then removed.

Furthermore, document DD 201302 relates to the synthesis of peroxides in 1, 1,2-trichloro-1,2,2-trifluoroethane. Once the synthesis has been carried out, the solvent is removed to obtain the concentrated peroxide.

In addition, documents U.S. Pat. Nos. 5,310,934 and 5,487,818 describe the purification of a peracid by dissolving it in an organic solvent. The solvent is subsequently removed and the peracid is in suspension in an aqueous solution.

However, a number of organic peroxides can not be stored or transported in the absence of solvent for safety reasons. Thus, the UN Orange book (20^{th} edition), concerning recommendations on the transport of hazardous materials, indicates that TBIC and TAIC can only be transported with at least 23% type A diluent. Consequently, these products are necessarily sold with a solvent. However, the presence of the solvent is disadvantageous in a certain number of applications, in particular for polymerization or crosslinking.

There is therefore a need to provide compositions of organic peroxides which are both stable and do not pose a risk to safety, and which can be advantageously used in various applications, in particular for polymerization or crosslinking.

### SUMMARY OF THE INVENTION

The invention relates firstly to a process for preparing a mixture comprising an organic peroxide and an additional compound, the process comprising the steps of:
a) bringing:
   a first composition comprising said organic peroxide and a solvent, into contact with
   a second composition comprising said additional compound, and
b) removing the solvent from the third composition thus obtained;
the additional compound being chosen from the group consisting of a monomer, an additional solvent and an additional organic peroxide.

The invention also relates to a mixture comprising an organic peroxide and an additional compound that can be obtained by the process according to any one of the preceding claims, preferably chosen from the group consisting of:
a mixture of TAIC and ADC,
a mixture of TBIC and ADC,
a mixture of 1,1-di-(tert-amylperoxy)cyclohexane and ADC,
a mixture of TAIC and TBEC,
a mixture of TBIC and TBEC,
a mixture of TAIC and TAEC, and
a mixture of TBIC and TAEC.

The invention also relates to a process for polymerization using a mixture comprising an organic peroxide and an additional compound, comprising the steps of:
a) optionally, bringing said mixture into contact with at least one monomer to be polymerized, in particular when the additional compound is not a monomer; and
b) polymerizing said at least one monomer.

The invention also relates to a process for crosslinking a polymer using a mixture comprising an organic peroxide and an additional compound, comprising the steps of:
a) optionally, bringing said mixture into contact with a polymer composition; and
b) crosslinking said polymer by means of said mixture.

The present invention makes it possible to satisfy the need expressed above. It provides more particularly compositions of organic peroxides which are both stable and do not pose a risk to safety, and which can be advantageously used in various applications, in particular for polymerization or crosslinking.

This is accomplished by preparing an organic peroxide in a solvent, combining the composition comprising said organic peroxide and said solvent with an additional compound and also removing said solvent, in order to obtain a mixture comprising said peroxide and said additional compound in the absence of solvent while limiting the dangers associated with handling organic peroxides.

### DESCRIPTION OF EMBODIMENTS OF THE INVENTION

The invention is now described in greater detail and in a nonlimiting manner in the description that follows.

### Preparation Process

The invention relates to a process for preparing a mixture comprising an organic peroxide and an additional compound, the process comprising the steps of:
a) bringing:
   a first composition comprising said organic peroxide and a solvent, into contact with
   a second composition comprising said additional compound, and
b) removing the solvent from the third composition thus obtained;
the additional compound being chosen from the group consisting of a monomer, an additional solvent and an additional organic peroxide.

Preferably, said organic peroxide is an unstable product during synthesis. Thus, for safety reasons, said peroxide needs to be diluted and/or synthesized in a solvent.

Preferably, the organic peroxide is chosen from the group consisting of OO-tert alkyl O-alkyl monoperoxycarbonate, peroxyketals, peroxyesters and percarbonates.

Preferably, the organic peroxide is not a hydroperoxide or an acid peroxide.

The organic peroxide may preferably be chosen from the group consisting of OO-tert-alkyl O-alkyl monoperoxycarbonate, diisopropyl peroxycarbonate, polyether poly(tert-butyl peroxycarbonate), tert-amyl peroxypivalate, tert-butyl peroxypivalate, 2,2-di-(tert-butylperoxy)butane, 2,2-di-(tert-amylperoxy)butane, 1,1-di-(tert-butylperoxy)-3,3,5-trimethylcyclohexane, 1,1-di-(tert-butylperoxy)cyclohexane, 1, 1-di-(tert-amylperoxy)cyclohexane and combinations thereof.

The organic peroxide can in particular be an OO-tert-alkyl O-alkyl monoperoxycarbonate, preferably chosen from the group consisting of: OO-tert-butyl O-2-isopropyl monoperoxycarbonate (TBIC) and OO-tert-amyl O-2-isopropyl monoperoxycarbonate (TAIC). These monoperoxycarbonates are commercially sold by Arkema under the name Luperox® or Lupersol®.

In some embodiments, the first composition comprises at least two organic peroxides (especially combinations of the examples of organic peroxides above). Preferably, the first composition comprises a single organic peroxide. The solvent can in particular be chosen from the group consisting of a hydrocarbon, a chlorinated hydrocarbon and a fluorinated hydrocarbon and combinations thereof.

When the solvent is a hydrocarbon, the latter can in particular be chosen from the group consisting of pentane, cyclopentane, hexane, cyclohexane, heptane, octane, decane, dodecane, isododecane, methylbutane, methylpentane and dimethylbutane, preferably is chosen from the group consisting of pentane, cyclopentane, hexane, cyclohexane, heptane and octane, and more preferentially chosen from the group consisting of pentane, cyclohexane and heptane, and more preferentially is heptane.

When the solvent is a chlorinated hydrocarbon, the latter can in particular be chosen from the group consisting of: methylene chloride, chloroform, tetrachloromethane, tetrachlorethylene, trichlorethylene, 1,1, 1-trichloroethane and 1,1,2-trichloroethane, preferably is chosen from the group consisting of methylene chloride, tetrachloromethane and tetrachlorethylene.

When the solvent is a fluorinated hydrocarbon, the latter can in particular be chosen from the group consisting of 1,1,2-trichloro-1,2,2-trifluoroethane, 1,1,2,2-tetrachloro-1,2-difluoroethane, 1,1, 1,3,3-pentafluorobutane and 1,1,1,3,3-pentafluoropropane, preferably is chosen from the group consisting of 1,1,1,3,3-pentafluorobutane and 1,1,1,3,3-pentafluoropropane.

Preferably, said solvent advantageously fulfills one, preferably two, more preferentially three and even more preferentially all of the following conditions:
be inert with respect to the organic peroxide, the additional compound and the reagents used for the synthesis of the organic peroxide;
not be reactive under the conditions of synthesis of the organic peroxide, in particular be inert with respect to the acids or bases used for the synthesis of the organic peroxide;
have a boiling point compatible with the stability of the organic peroxide, that is to say preferably at least 20° C. lower, preferably at least 30° C. lower, more preferentially at least 40° C. lower than the half-life temperature at one hour (HLT at 1 h) of said organic peroxide;
have a boiling point lower than the boiling point of the additional compound. For example, the boiling point of said solvent is at least 5° C. lower than that of the additional compound, preferably at least 10° C. lower, more preferentially at least 15° C. lower and more preferentially at least 20° C. lower than that of the additional compound.

In some embodiments, at least two solvents can be present in the first composition.

When at least two solvents are present in the first composition, only one, at least two or all of said at least two solvents can be removed during step b).

Preferably, only one solvent is present in the first composition.

For the purposes of the present invention, the term “monomer” is preferably understood to mean a compound capable of polymerizing. Thus, said monomer can also be an oligomer.

When the additional compound is a monomer, it can in particular be chosen from the group consisting of a carbonate and an ester.

Preferably, the carbonate is chosen from the group consisting of diethylene glycol bis(allyl carbonate) (ADC), ethylene glycol divinyl carbonate (CAS134073-18-0) and glycerin tris (vinyl carbonate) (CAS134073-25-9); it is preferably diethylene glycol bis(allyl carbonate) (ADC).

Preferably, the ester is 2,5,8, 11-tetraoxa-dodecanedioic acid, diethylenyl ester (CAS134073-19-1)

Preferably, the mixture comprising the organic peroxide and the monomer comprises TAIC and ADC, and preferably consists essentially of TAIC and ADC, and more preferably consists of TAIC and ADC.

Alternatively, the mixture comprising the organic peroxide and the monomer comprises TBIC and ADC, and preferably consists essentially of TBIC and ADC, and more preferably consists of TBIC and ADC.

Alternatively, the mixture comprising the organic peroxide and the monomer comprises 1,1-di-(tert-amylperoxy)cyclohexane and ADC, and preferably consists essentially of 1,1-di-(tert-amylperoxy)cyclohexane and ADC, and more preferably consists of 1,1-di-(tert-amylperoxy)cyclohexane and ADC.

The abovementioned mixtures cannot be obtained by direct synthesis of the organic peroxide in ADC because of the decomposition/reaction of the latter under the synthesis conditions.

Preferably, when the additional compound is a monomer, the organic peroxide can be a polymerization initiator for said monomer.

When the additional compound is an additional solvent, that is to say a solvent other than the solvent or than one of the solvents present in the first composition, it may in particular be a reactive solvent, that is to say a solvent which is capable of reacting with at least one compound present during the synthesis of the organic peroxide—and which therefore cannot be easily used during this synthesis.

Preferably, the additional solvent is not water.

The additional solvent can be chosen in particular from the group consisting of esters (ethyl acetate, butyl acetate, etc.), diesters such as alkyl phthalates (diethylhexyl phthalates, dimethyl phthalate, dicyclohexyl phthalates, etc.) (such as those mentioned in application WO 2004/000799), malonates, adipates, maleic anhydrides and esters, fumarates, cinnamates, stilbene (see in particular document U.S. Pat. No. 4,131,728), olefins, in particular α-olefins (1-hexene, 1-heptene, 1-octene, 1-nonene, 1-decene, 1-undecene, 1-dodecene) and cyclic alkenes (cycloheptene, cyclooctene, cyclododecene).

The term “solvent” herein denotes a substance in which the organic peroxide can be dissolved.

When the additional compound is an additional organic peroxide, it can be chosen in particular from the group consisting of OO-tert-butyl O-(2-ethylhexyl) monoperoxycarbonate (TBEC), OO-tert-butyl O-(2-isopropyl) monoperoxycarbonate (TBIC), OO-tert-amyl O-2-(ethylhexyl) monoperoxycarbonate (TAEC), and OO-tert-amyl O-(2-isopropyl) monoperoxycarbonate (TAIC), tert-amyl peroxypivalate (Luperox® 554), tert-butyl peroxypivalate (Luperox® 11) or polyether poly-(tert-butyl) peroxycarbonate (Luperox® JWEB50), diisopropyl peroxycarbonate, 2,2-di-(tert-butylperoxy)butane, 2,2-di-(tert-amylperoxy)butane, 1,1-di-(tert-butylperoxy)-3,3,5-trimethylcyclohexane, 1,1-di-(tert-butylperoxy)cyclohexane, and 1,1-di-(tert-amylperoxy)cyclohexane, and combinations thereof.

It is understood that the additional organic peroxide is different than the organic peroxide present in the first composition.

According to one aspect of the invention, the mixture comprising the organic peroxide and the additional organic peroxide comprises TAIC and TBEC, and preferably consists essentially of TAIC and TBEC, and more preferably consists of TAIC and TBEC.

Alternatively, the mixture comprising the organic peroxide and the additional organic peroxide comprises TBIC and TBEC, and preferably consists essentially of TBIC and TBEC, and more preferably consists of TBIC and TBEC.

Alternatively, the mixture comprising the organic peroxide and the additional organic peroxide comprises TAIC and TAEC, and preferably consists essentially of TAIC and TAEC, and more preferably consists of TAIC and TAEC.

Alternatively, the mixture comprising the organic peroxide and the additional organic peroxide comprises TBIC and TAEC, and preferably consists essentially of TBIC and TAEC, and more preferably consists of TBIC and TAEC.

Preferably, the mixture is chosen from the group consisting of:
OO-tert-amyl O-2-isopropyl monoperoxycarbonate and diethylene glycol bis(allyl carbonate),
OO-tert-butyl O-2-isopropyl monoperoxycarbonate and diethylene glycol bis(allyl carbonate),
OO-tert-amyl O-2-isopropyl monoperoxycarbonate and OO-tert-butyl O-(2-ethylhexyl) monoperoxycarbonate,
OO-tert-butyl O-2-isopropyl monoperoxycarbonate and OO-tert-butyl O-(2-ethylhexyl) monoperoxycarbonate,
OO-tert-amyl O-(2-isopropyl) monoperoxycarbonate and OO-tert-amyl O-(2-ethylhexyl) monoperoxycarbonate, and
OO-tert-butyl O-(2-isopropyl) monoperoxycarbonate and OO-tert-amyl O-(2-ethylhexyl) monoperoxycarbonate.

The abovementioned mixtures cannot be obtained by conventional synthesis, since complex mixtures are formed due to transperoxidation reactions.

In some embodiments, several additional compounds may be present in the mixture.

Preferably, the second composition is not water, nor an aqueous solution.

The first composition can in particular be obtained by synthesis of said organic peroxide in said solvent.

Thus, preferably, the process according to the invention comprises a step a′), prior to step a), of synthesizing a first composition comprising said organic peroxide and a solvent as defined above.

In some embodiments, the step of synthesizing the organic peroxide comprises the synthesis of two or more organic peroxides in the solvent. Preferably, a single organic peroxide is synthesized in the solvent.

The methods of synthesizing organic peroxides are well known to those skilled in the art and are for example described in scientific works such as “Organic peroxides” publisher Swern, 1971, “Organic peroxides” publisher Ando, 1992 or “Les peroxides organiques” [“Organic peroxides”] by V. Karnojitzki Hermann editions, 1958.

The general methods of synthesizing organic peroxides consist in mixing several ingredients, simultaneously or in a certain order. Depending on the peroxide to be synthesized, an alcohol, an olefin, a ketone, an acid chloride, a chloroformate, and optionally a solvent, are mixed with aqueous hydrogen peroxide or a hydroperoxide in the presence of an acid solution (sulfuric acid, sulfonic acid, perchloric acid, etc.) or a basic solution (NaOH, KOH, etc.) while controlling the reaction temperature. After synthesis and decanting, the organic peroxide is washed, depending on the product, with a basic solution (NaOH, KOH, Na₂ CO₃, etc.), and/or a reducing solution (sulfite, etc.) and water.

The general method for preparing peroxydicarbonates (R1OC(O)OOC(O)OR2), and monoperoxycarbonates (R1OC(O)OOCR2) consists of the action of an acid chloroformate on the alkali salt respectively of aqueous hydrogen peroxide or of a hydroperoxide.

Stoichiometric amounts of acid chloroformate and base are added, simultaneously or one after the other, to the vigorously stirred aqueous hydrogen peroxide or hydroperoxide, while maintaining the temperature of the medium at the desired level. Once the addition is complete, the stirring of the medium is continued to complete the reaction.

Washing is performed with a basic solution (NaOH or Na₂CO₃) at 5-10% to remove traces of unreacted chloroformate, then with water until neutral. The product is then dried, for example over anhydrous Na₂SO₄.

Preferably, step a) of bringing the first composition into contact with the second composition is carried out by transferring the first composition into the second composition or by transferring the second composition into the first composition.

Preferably, said bringing into contact, in particular said transfer of the first composition into the second composition or of the second composition into the first composition, can be carried out using a syringe, a cannula, or a dropping funnel, or by simple transfer.

Preferably, step b) of removing the solvent is carried out by heating the composition obtained in step a).

Preferably, the heating in step b) is carried out at a temperature greater than or equal to the boiling point of the solvent of the first composition, preferably at a temperature between the boiling point of said solvent and a temperature 10° C. above the boiling point of said solvent, more preferentially at a temperature between the boiling point of said solvent and a temperature 5° C. above the boiling point of said solvent, preferably at the boiling point of said solvent.

Preferably, the heating step b) is chosen from the group consisting of vacuum distillation, air stripping and steam stripping.

Preferably, at least 50%, preferably at least 70%, more preferentially at least 80%, more preferentially at least 90%, more preferentially at least 95%, more preferentially at least 99% and more preferentially at least 99.9% of the solvent(s) of the first composition is removed in step b). Preferably, all of the solvent(s) of the first composition are removed in step b).

Preferably, the residual solvent after step b) of removing the solvent represents in particular less than 10 000 ppm, preferably less than 5000 ppm, more preferably less than 2000 ppm, more preferably less than 1000 ppm, more preferably less than 500 ppm, more preferably less than 300 ppm, more preferably less than 100 ppm, more preferably less than 50 ppm, more preferably less than 30 ppm, more preferably less than 10 ppm relative to the mixture comprising the organic peroxide and the additional compound (by mass content).

In certain embodiments, the mixture comprising the organic peroxide and the additional compound can be reactive under certain conditions, for example of temperature and/or pressure, and inert under other conditions, for example of temperature and/or pressure.

### Mixture

The present invention also relates to a mixture comprising an organic peroxide and an additional compound that can be obtained by the process according to the invention.

Said mixture, said organic peroxide and said additional compound are as defined above.

Preferably, said mixture comprises less than 10 000 ppm, preferably less than 5000 ppm, more preferably less than 2000 ppm, more preferably less than 1000 ppm, more preferably less than 500 ppm, more preferably less than 300 ppm, more preferably less than 100 ppm, more preferably less than 50 ppm, more preferably less than 30 ppm, more preferably less than 10 ppm of solvent (other than the additional compound) relative to the mixture comprising the organic peroxide and the additional compound (by mass content).

Preferably, the mixture comprising the organic peroxide and the monomer is chosen from the group consisting of:
a mixture of TAIC and ADC,
a mixture of TBIC and ADC,
a mixture of 1,1-di-(tert-amylperoxy)cyclohexane and ADC,
a mixture of TAIC and TBEC,
a mixture of TBIC and TBEC,
a mixture of TAIC and TAEC, and
a mixture of TBIC and TAEC.

Said mixture preferably comprises less than 10 000 ppm, preferably less than 5000 ppm, more preferably less than 2000 ppm, more preferably less than 1000 ppm, more preferably less than 500 ppm, more preferably less than 300 ppm, more preferably less than 100 ppm, more preferably less than 50 ppm, more preferably less than 30 ppm, more preferably less than 10 ppm of solvent (other than the additional compound) relative to the mixture comprising the organic peroxide and the additional compound (by mass content).

Preferably, said mixture comprises less than 2% by weight of water, preferably less than 1% by weight of water, preferably less than 0.5% by weight of water, and particularly preferably does not comprise water.

### Polymerization Process

The invention also relates to a process for polymerization using a mixture comprising the organic peroxide and the additional compound as defined above, comprising the steps of:
a) optionally, bringing said mixture into contact with at least one monomer to be polymerized, in particular when the additional compound is not a monomer; and
b) polymerizing said at least one monomer.

Said polymer production process may also include a step a′), prior to step a), of preparing said mixture with the preparation process according to the invention.

The invention also relates to the polymer that can be obtained by the polymerization process according to the invention.

### Crosslinking Process

The invention also relates to a process for crosslinking a polymer using a mixture comprising the organic peroxide and the additional compound as defined above, comprising the steps of:
a) optionally, bringing said mixture into contact with a polymer composition; and
b) crosslinking said polymer by means of said mixture.

Said crosslinking process may also include a step a′), prior to step a), of preparing said mixture with the preparation process according to the invention.

Said polymer may be chosen from the group consisting of an ethylene/propylene copolymer (EPM), an ethylene/propylene/diene terpolymer (EPDM), a butadiene/acrylonitrile copolymer (NBR), a hydrogenated butadiene/acrylonitrile copolymer (HNBR), fluorocarbon (FKM), silicone rubber (Q), polyethylene (PE) and an ethylene/vinyl acetate copolymer (EVA).

The invention also relates to the crosslinked polymer that can be obtained by the crosslinking process according to the invention.

### EXAMPLES

The examples that follow illustrate the invention without limiting it.

### Example 1: Preparation of TAIC/ADC

First, TAIC is synthesized in heptane:

At 15° C., 26 g of 85% w/w tert-amyl hydroperoxide (TAHP) solution, 14 g of heptane and 56 g of 25% w/w potassium hydroxide solution are mixed. The mixture is cooled to 0° C. with stirring. 24.6 g of isopropyl chloroformate are then added while maintaining the temperature between 0° C. and 3° C. for 20 min. After the addition of isopropyl chloroformate, the medium is maintained between 0° C. and 3° C. for 100 min. The solution is decanted at 15° C. for 10 min and 50 g of organic phase are recovered. The organic phase is then washed several times with basic solutions and water. After the washing operations, 47 g of organic phase containing 69% w/w of TAIC and 30% w/w of heptane are recovered.

14 g of synthetic organic phase containing TAIC are subsequently mixed with 40 g of ADC in the flask of a rotary evaporator. The flask is heated to 45° C. under vacuum (110 mbar) and the condenser is maintained at 0° C. After a few minutes, the boiling stops and the pressure is lowered to 10 mbar. After 15 min, all the heptane solvent is recovered in the condenser and 50 g of a solution containing 20% w/w of TAIC in ADC are recovered in the flask of the rotary evaporator.

### Example 2: Preparation of TAIC in TBEC

First, TAIC is synthesized in heptane as above.

A portion of the solution is mixed with TBEC: Thus, 20 g of synthetic organic phase containing TAIC is mixed with 42 g of TBEC in the flask of a rotary evaporator. The flask is heated to 45° C. under vacuum (110 mbar) and the condenser is maintained at 0° C. After a few minutes, the boiling stops and the pressure is lowered to 10 mbar. After 15 min, all the heptane solvent is recovered in the condenser and 56 g of a solution containing 24% w/w of TAIC and 75% of TBEC are recovered in the flask of the rotary evaporator.

### Example 3: Preparation of 1,1-di-(tert-amylperoxy)cyclohexane in ADC

48 g of an 85% w/w solution of tert-amyl hydroperoxide (TAHP), 18 g of 99% w/w cyclohexanone and 17 g of 99% w/w pentane are mixed, at a temperature of 10° C., in a 300 cc reactor. The mixture is then cooled to −10° C. with stirring. Then 29 g of 70% w/w sulfuric acid are added while maintaining the temperature between-10° C. and 0° C. for 45 min. After the addition of sulfuric acid, the temperature is raised and maintained at 15-16° C. for 180 min. The solution is then decanted at 15-16° C. for 60 min and 66 g of organic phase are recovered. The organic phase is then washed several times with a 10% w/w solution of sodium sulfite, then an 8% w/w solution of sodium bicarbonate and finally demineralized water. After the washing operations, 54 g of organic phase containing 85% w/w of 1,1-di-(tert-amylperoxy)cyclohexane are recovered.

50 g of organic phase containing the 1,1-di-(tert-amylperoxy) cyclohexane are then mixed with 365 g of ADC in the flask of a rotary evaporator. The flask is heated to 40° C. under a vacuum to 5 mbar. When the boiling stops, the apparatus is returned to atmospheric pressure and 407 g of a solution of 1,1-di-(tert-amylperoxy)cyclohexane in ADC containing 10% w/w of organic peroxide are recovered.

### Example 4: Preparation of TAIC in TBEC (Counterexample)

TAIC is prepared directly in TBEC: 15.8 g of a 25% w/w potassium hydroxide solution are mixed with 7.8 g of an 85% w/w TAHP solution and 20.7 g of TBEC. 8.6 g of an isopropyl chloroformate solution are gradually added to this solution while maintaining the temperature of the medium from 10 to 15° C. The reaction is then maintained at 15° C. for 1 hour. After decanting and washing operations, 37.6 g of organic phase containing 19.7% of TAIC, 7.8% of TBIC, 14.7% of TAEC and 43% of TBEC are obtained.

A mixture of four different peroxides, which thus does not correspond to the desired binary mixture, is thus obtained when the TAIC is prepared directly in the TBEC.

1-14. (canceled)

15. A process for preparing a mixture comprising an organic peroxide and an additional compound, the process comprising the steps of:
a) bringing:
a first composition comprising said organic peroxide and a solvent, into contact with
a second composition comprising said additional compound, and
b) removing the solvent from a third composition thus obtained;
the additional compound being chosen from the group consisting of a monomer, an additional solvent, and an additional organic peroxide.

16. The process as claimed in claim 15, wherein said first composition is obtained by synthesizing said organic peroxide in said solvent.

17. The process as claimed in claim 15, wherein said organic peroxide is selected from the group consisting of OO-tert-alkyl O-alkyl monoperoxycarbonate, diisopropyl peroxycarbonate, polyether poly(tert-butyl peroxycarbonate), tert-amyl peroxypivalate, tert-butyl peroxypivalate, 2,2-di-(tert-butylperoxy)butane, 2,2-di-(tert-amylperoxy)butane, 1,1-di-(tert-butylperoxy)-3,3,5-trimethylcyclohexane, 1,1-di-(tert-butylperoxy)cyclohexane, 1,1-di-(tert-amylperoxy)cyclohexane and combinations thereof.

18. The process as claimed in claim 15, wherein the solvent of the first composition is selected from the group consisting of a hydrocarbon, a chlorinated hydrocarbon, a fluorinated hydrocarbon, and combinations thereof.

19. The process as claimed in claim 15, wherein the additional compound is a monomer selected from the group consisting of a carbonate and an ester.

20. The process as claimed in claim 15, wherein the additional compound is a monomer and the organic peroxide is a polymerization initiator for said monomer.

21. The process as claimed in claim 15, wherein the additional compound is an additional solvent.

22. The process as claimed in claim 15, wherein the additional compound is an additional organic peroxide.

23. The process as claimed in claim 19, wherein the mixture is selected from the group consisting of:
OO-tert-amyl O-(2-isopropyl) monoperoxycarbonate and diethylene glycol bis(allyl carbonate),
OO-tert-butyl O-(2-isopropyl) monoperoxycarbonate and diethylene glycol bis(allyl carbonate),
1,1-di-(tert-amylperoxy) cyclohexane and diethylene glycol bis(allyl carbonate),
OO-tert-amyl O-(2-isopropyl) monoperoxycarbonate and OO-tert-butyl O-(2-ethylhexyl) monoperoxycarbonate,
OO-tert-butyl O-(2-isopropyl) monoperoxycarbonate and OO-tert-butyl O-(2-ethylhexyl) monoperoxycarbonate,
OO-tert-amyl O-(2-isopropyl) monoperoxycarbonate and OO-tert-amyl O-(2-ethylhexyl) monoperoxycarbonate, and
OO-tert-butyl O-(2-isopropyl) monoperoxycarbonate and OO-tert-amyl O-(2-ethylhexyl) monoperoxycarbonate.

24. The process as claimed in claim 15, wherein step b) is carried out by heating the composition obtained in step a), by a process selected from the group consisting of vacuum distillation, air stripping, and steam stripping.

25. A mixture comprising an organic peroxide and an additional compound that can be obtained by the process according to claim 15, wherein the mixture is selected from the group consisting of:
a mixture of TAIC and ADC,
a mixture of TBIC and ADC,
a mixture of 1,1-di-(tert-amylperoxy)cyclohexane and ADC,
a mixture of TAIC and TBEC,
a mixture of TBIC and TBEC,
a mixture of TAIC and TAEC, and
a mixture of TBIC and TAEC.

26. The mixture as claimed in claim 25, comprising less than 10,000 ppm of solvent other than the additional compound relative to the mixture comprising the organic peroxide and the additional compound.

27. A process for polymerizing a monomer comprising
a) bringing a mixture comprising an organic peroxide and an additional compound into contact with the monomer to be polymerized; and
b) polymerizing said monomer, wherein the mixture is selected from the group consisting of:
a mixture of TAIC and ADC,
a mixture of TBIC and ADC,
a mixture of 1,1-di-(tert-amylperoxy)cyclohexane and ADC,
a mixture of TAIC and TBEC,
a mixture of TBIC and TBEC,
a mixture of TAIC and TAEC, and
a mixture of TBIC and TAEC.

28. The process as claimed in claim 19, wherein the carbonate is selected from the group consisting of diethylene glycol bis(allyl carbonate), ethylene glycol divinyl carbonate and glycerin tris(vinyl carbonate), and the ester is 2,5,8,11-tetraoxa-dodecanedioic acid, diethylenyl ester.

29. The process as claimed in claim 21, wherein the additional compound is not water.

30. The process as claimed in claim 21, wherein the additional compound is selected from the group consisting of esters, diesters, malonates and adipates, maleic anhydrides and esters, fumarates, cinnamates, stilbene, olefins, and cyclic alkenes.

31. The process as claimed in claim 22, wherein the additional organic peroxide is selected from the group consisting of OO-tert-butyl O-(2-ethylhexyl) monoperoxycarbonate, OO-tert-butyl O-(2-isopropyl) monoperoxycarbonate, OO-tert-amyl O-(2-ethylhexyl) monoperoxycarbonate, and OO-tert-amyl O-(2-isopropyl) monoperoxycarbonate, tert-amyl peroxypivalate, tert-butyl peroxypivalate or polyether poly-(tert-butyl) peroxycarbonate, diisopropyl peroxycarbonate, 2,2-di-(tert-butylperoxy)butane, 2,2-di-(tert-amylperoxy)butane, 1,1-di-(tert-butylperoxy)-3,3,5-trimethylcyclohexane, 1,1-di-(tert-butylperoxy)cyclohexane, and 1,1-di-(tert-amylperoxy)cyclohexane, and combinations thereof.
